(19) United States
(12) Patent Application Publication
(10) Pub. No.: US 2003/0003450 A1
(43) Pub. Date: Jan. 2, 2003

(54) Computer software products for gene expression analysis using linear programming

(75) Inventor: Earl Hubbell, Los Angeles, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, Inc.

(21) Appl. No.: 09/746,036

(22) Filed: Dec. 21, 2000

(51) Int. Cl. C12Q 1/68; G06F 19/00; G01N 33/48; G01N 33/50

(52) U.S. Cl. 435/6; 702/20

(57) ABSTRACT

Methods and computer software products are provided for analyzing gene expression data. In one embodiment, linear programming is used to estimate relative transcripts. Bootstrapping methods are used to obtain confidence interval for estimators.

### FIELD OF INVENTION

This invention is related to bioinformatics and biological data analysis. Specifically, this invention provides methods, computer software products and systems for the analysis of biological data.

### BACKGROUND OF THE INVENTION

Many biological functions are carried out by regulating the expression levels of various genes, either through changes in the copy number of the genetic DNA, through changes in levels of transcription (e.g. through control of initiation, provision of RNA precursors, RNA processing, etc.) of particular genes, or through changes in protein synthesis. For example, control of the cell cycle and cell differentiation, as well as diseases, are characterized by the variations in the transcription levels of a group of genes.

Recently, massive parallel gene expression monitoring methods have been developed to monitor the expression of a large number of genes using nucleic acid array technology which was described in detail in, for example, U.S. Pat. No. 5,871,928; de Saizieu, et al., 1998, Bacteria Transcript Imaging by Hybridization of total RNA to Oligonucleotide Arrays, Nature Biotechnology, 16:45-48; Wodicka et al., 1997, Genome-wide Expression Monitoring in Saccharomyces cerevisiae, Nature Biotechnology 15:1359-1367; Lockhart et al., 1996, Expression Monitoring by Hybridization to High Density Oligonucleotide Arrays. Nature Biotechnology 14:1675-1680; Lander, 1999, Array of Hope, Nature-Genetics, 21(suppl.), at 3.

Massive parallel gene expression monitoring experiments generate unprecedented amounts of information. For example, a commercially available GeneChip® array set is capable of monitoring the expression levels of approximately 6,500 murine genes and expressed sequence tags (ESTs) (Affymetrix, Inc, Santa Clara, Calif., USA). Array sets for approximately 60,000 human genes and EST clusters, 24,000 rat transcripts and EST clusters and arrays for other organisms are also available from Affymetrix. Effective analysis of the large amount of data may lead to the development of new drugs and new diagnostic tools. Therefore, there is a great demand in the art for methods for organizing, accessing and analyzing the vast amount of information collected using massive parallel gene expression monitoring methods.

### SUMMARY OF THE INVENTION

The current invention provides methods, systems and computer software products suitable for analyzing data from gene expression monitoring experiments that employ multiple probes against a single target.

In one aspect of the invention, methods, systems and computer software products are provided for gene expression data analysis. The methods are based on constraining possible expression levels using simple models.

The embodiments of the invention are particularly useful for analyzing results of nucleic acid probe array based gene expression experiments where probes generally hybridize linearly with their targets; where the major error is cross hybridization; where hybridization intensities are positive and continuous quantities; where relative few probe suffer death, saturation, or irregular noise and where chip effects are multiplicative changes to the scale of the intensities. In such embodiments, the intensity (I) of a probe may be decomposed to: I=S·C·T+H, where: S is chip scale (to adjust for variations among chips); C is coupling between the level of the targeted transcript in the sample and the intensity; T is the relative level of the transcript; and H is the effect of cross hybridization. While effect of cross hybridization on intensity is generally unknown, it is greater than zero. Therefore: I≧S·C·T or log(I)≧log(S)+log(C)+log(T). Linear programming may be used to maximize the true effect and obtain estimates of the parameters including T, the relative level of the transcript in a sample.

In some embodiments, the methods of the invention include steps of obtaining a plurality of intensities, each of which reflects the hybridization of one of a plurality of probes in the plurality of samples; and determining the couplings between the level of the transcript and the intensities, relative transcript levels and scales of probe arrays by minimizing the effect of cross-hybridization and maximing true effects using linear programming with the constraint that the effect of cross-hybridization is non-zero. The minimizing step may be performed by maximizing Σ(s(i)+c(j,k)+x(k,l)) or minimizing Σ(Y(i,j,k,l)−s(i)−c(j,k)−x(k,l)) with the constraint Y(i,j,k,l)≧s(i)+c(j,k)+x(k,l), wherein s(i) is log(scale of probe array) for the ith probe array, c(j,k) is the (log(the coupling between transcript and intensity)) for jth probe and kth transcript, x(k,l) is the log(relative transcript level) for the kth transcript in the lth sample, and Y(i,j,k,l) is the log(I) for jth probe for kth transcript in the ith probe array hybridized with the lth sample. Because Σx(k,l)=0, Σ(s(i)+c(j,k)+x(k,l)) is equivalent to Σ(s(i)+c(j,k)) . In some embodiments, the maximizing or minimizing is further constrained by the condition that coupling for perfect match probes is greater than that for mismatch probes. In preferred embodiments, the scale of probe array is determined independent of the maximizing, such as using normalization probes on the probe arrays.

In some preferred embodiments, methods are provided to determine confidence intervals of estimators such as the relative transcript levels, couplings and scales by bootstrapping on residues, probe arrays or probes.

Some embodiments of the system include a processor; and a memory being coupled with the processor; the memory storing a plurality of machine instructions that cause the processor to perform a method steps of the invention when implemented by the processor.

Computer software products of the invention may include a computer readable medium having computer executable instructions for performing the methods of the invention.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 illustrates an example of a computer system that may be utilized to execute the software of an embodiment of the invention.

FIG. 2 illustrates a system block diagram of the computer system of FIG. 1.

FIG. 3 shows one embodiment of the gene expression analysis method of the invention.

### DETAILED DESCRIPTION OF THE PREFERRED EMBODIMENTS

Reference will now be made in detail to the preferred embodiments of the invention. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention. All cited references, including patent and non-patent literature, are incorporated herein by reference in their entireties for all purposes.

I. Gene Expression Monitoring With High Density Oligonucleotide Probe Arrays

High density nucleic acid probe arrays, also referred to as “DNA Microarrays,” have become a method of choice for monitoring the expression of a large number of genes. As used herein, “Nucleic acids” may include any polymer or oligomer of nucleosides or nucleotides (polynucleotides or oligonucleotidies), which include pyrimdine and purine bases, preferably cytosine, thymine, and uracil, and adenine and guanine, respectively. See Albert L. Lehninger, PRINCIPLES OF BIOCHEMISTRY, at 793-800 (Worth Pub. 1982) and L. Stryer BIOCHEMISTRY, 4^{th} Ed., (March 1995), both incorporated by reference. “Nucleic acids” may include any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

“A target molecule” refers to a biological molecule of interest. The biological molecule of interest can be a ligand, receptor, peptide, nucleic acid (oligonucleotide or polynucleotide of RNA or DNA), or any other of the biological molecules listed in U.S. Pat. No. 5,445,934 at col. 5, line 66 to col. 7, line 51. For example, if transcripts of genes are the interest of an experiment, the target molecules would be the transcripts. Other examples include protein fragments, small molecules, etc. “Target nucleic acid” refers to a nucleic acid (often derived from a biological sample) of interest. Frequently, a target molecule is detected using one or more probes. As used herein, a “probe” is a molecule for detecting a target molecule. It can be any of the molecules in the same classes as the target referred to above. A probe may refer to a nucleic acid, such as an oligonucleotide, capable of binding to a target nucleic acid of complementary sequence through one or more types of chemical bonds, usually through complementary base pairing, usually through hydrogen bond formation. As used herein, a probe may include natural (i.e. A, G, U, C, or T) or modified bases (7-deazaguanosine, inosine, etc.). In addition, the bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages. Other examples of probes include antibodies used to detect peptides or other molecules, any ligands for detecting its binding partners. When referring to targets or probes as nucleic acids, it should be understood that there are illustrative embodiments that are not to limit the invention in any way.

In preferred embodiments, probes may be immobilized on substrates to create an array. An “array” may comprise a solid support with peptide or nucleic acid or other molecular probes attached to the support. Arrays typically comprise a plurality of different nucleic acids or peptide probes that are coupled to a surface of a substrate in different, known locations. These arrays, also described as “microarrays” or colloquially “chips” have been generally described in the art, for example, in Fodor et al., Science, 251:767-777 (1991), which is incorporated by reference for all purposes. Methods of forming high density arrays of oligonucleotides, peptides and other polymer sequences with a minimal number of synthetic steps are disclosed in, for example, 5,143,854, 5,252,743, 5,384,261, 5,405,783, 5,424,186, 5,429,807, 5,445,943, 5,510,270, 5,677,195, 5,571,639, 6,040,138, all incorporated herein by reference for all purposes. The oligonucleotide analogue array can be synthesized on a solid substrate by a variety of methods, including, but not limited to, light-directed chemical coupling, and mechanically directed coupling. See Pirrung et al., U.S. Pat. No. 5,143,854 (see also PCT Application No. WO 90/15070) and Fodor et al., PCT Publication Nos. WO 92/10092 and WO 93/09668, U.S. Pat. Nos. 5,677,195, 5,800,992 and 6,156,501 which disclose methods of forming vast arrays of peptides, oligonucleotides and other molecules using, for example, light-directed synthesis techniques. See also, Fodor et al., Science, 251, 767-77 (1991). These procedures for synthesis of polymer arrays are now referred to as VLSIPS™ procedures. Using the VLSIPS™ approach, one heterogeneous array of polymers is converted, through simultaneous coupling at a number of reaction sites, into a different heterogeneous array. See, U.S. Pat. Nos. 5,384,261 and 5,677,195.

Methods for making and using molecular probe arrays, particularly nucleic acid probe arrays are also disclosed in, for example, U.S. Pat. Nos. 5,143,854, 5,242,974, 5,252,743, 5,324,633, 5,384,261, 5,405,783, 5,409,810, 5,412,087, 5,424,186, 5,429,807, 5,445,934, 5,451,683, 5,482,867, 5,489,678, 5,491,074, 5,510,270, 5,527,681, 5,527,681, 5,541,061, 5,550,215, 5,554,501, 5,556,752, 5,556,961, 5,571,639, 5,583,211, 5,593,839, 5,599,695, 5,607,832, 5,624,711, 5,677,195, 5,744,101, 5,744,305, 5,753,788, 5,770,456, 5,770,722, 5,831,070, 5,856,101, 5,885,837, 5,889,165, 5,919,523, 5,922,591, 5,925,517, 5,658,734, 6,022,963, 6,150,147, 6,147,205, 6,153,743, 6,140,044 and D430024, all of which are incorporated by reference in their entireties for all purposes. Typically, a nucleic acid sample is a labeled with a signal moiety, such as a fluorescent label. The sample is hybridized with the array under appropriate conditions. The arrays are washed or otherwise processed to remove non-hybridized sample nucleic acids. The hybridization is then evaluated by detecting the distribution of the label on the chip. The distribution of label may be detected by scanning the arrays to determine florescence intensities distribution. Typically, the hybridization of each probe is reflected by several pixel intensities. The raw intensity data may be stored in a gray scale pixel intensity file. The GATC™ Consortium has specified several file formats for storing array intensity data. The final software specification is available at www.gatcconsortium.org and is incorporated herein by reference in its entirety. The pixel intensity files are usually large. For example, a GATC™ compatible image file may be approximately 50 Mb if there are about 5000 pixels on each of the horizontal and vertical axes and if a two byte integer is used for every pixel intensity. The pixels may be grouped into cells (see, GATC™ software specification). The probes in a cell are designed to have the same sequence (i.e., each cell is a probe area). A CEL file contains the statistics of a cell, e.g., the 75 percentile and standard deviation of intensities of pixels in a cell. The 75 percentile of pixel intensity of a cell is often used as the intensity of the cell. Methods for signal detection and processing of intensity data are additionally disclosed in, for example, U.S. Pat. Nos. 5,547,839, 5,578,832, 5,631,734, 5,800,992, 5,856,092, 5,936,324, 5,981,956, 6,025,601, 6,090,555, 6,141,096, 6,141,096, and 5,902,723. Methods for array based assays, computer software for data analysis and applications are additionally disclosed in, e.g., U.S. Pat. No. 5,527,670, 5,527,676, 5,545,531, 5,622,829, 5,631,128, 5,639,423, 5,646,039, 5,650,268, 5,654,155, 5,674,742, 5,710,000, 5,733,729, 5,795,716, 5,814,450, 5,821,328, 5,824,477, 5,834,252, 5,834,758, 5,837,832, 5,843,655, 5,856,086, 5,856,104, 5,856,174, 5,858,659, 5,861,242, 5,869,244, 5,871,928, 5,874,219, 5,902,723, 5,925,525, 5,928,905, 5,935,793, 5,945,334, 5,959,098, 5,968,730, 5,968,740, 5,974,164, 5,981,174, 5,981,185, 5,985,651, 6,013,440, 6,013,449, 6,020,135, 6,027,880, 6,027,894, 6,033,850, 6,033,860, 6,037,124, 6,040,138, 6,040,193, 6,043,080, 6,045,996, 6,050,719, 6,066,454, 6,083,697, 6,114,116, 6,114,122, 6,121,048, 6,124,102, 6,130,046, 6,132,580, 6,132,996, 6,136,269 and attorney docket numbers 3298.1 and 3309, all of which are incorporated by reference in their entireties for all purposes.

Nucleic acid probe array technology, use of such arrays, analysis array based experiments, associated computer software, composition for making the array and practical applications of the nucleic acid arrays are also disclosed, for example, in the following U.S. patent application Ser. Nos. 07/838,607, 07/883,327, 07/978,940, 08/030,138, 08/082,937, 08/143,312, 08/327,522, 08/376,963, 08/440,742, 08/533,582, 08/643,822, 08/772,376, 09/013,596, 09/016,564, 09/019,882, 09/020,743, 09/030,028, 09/045,547, 09/060,922, 09/063,311, 09/076,575, 09/079,324, 09/086,285, 09/093,947, 09/097,675, 09/102,167, 09/102,986, 09/122,167, 09/122,169, 09/122,216, 09/122,304, 09/122,434, 09/126,645, 09/127,115, 09/132,368, 09/134,758, 09/138,958, 09/146,969, 09/148,210, 09/148,813, 09/170,847, 09/172,190, 09/174,364, 09/199,655, 09/203,677, 09/256,301, 09/285,658, 09/294,293, 09/318,775, 09/326,137, 09/326,374, 09/341,302, 09/354,935, 09/358,664, 09/373,984, 09/377,907, 09/383,986, 09/394,230, 09/396,196, 09/418,044, 09/418,946, 09/420,805, 09/428,350, 09/431,964, 09/445,734, 09/464,350, 09/475,209, 09/502,048, 09/510,643, 09/513,300, 09/516,388, 09/528,414, 09/535,142, 09/544,627, 09/620,780, 09/640,962, 09/641,081, 09/670,510, 09/685,011, and 09/693,204 and in the following Patent Cooperative Treaty (PCT) applications/publications: PCT/NL90/00081, PCT/GB91/00066, PCT/US91/08693, PCT/US91/09226, PCT/US91/09217, WO/93/10161, PCT/US92/10183, PCT/GB93/00147, PCT/US93/01152, WO/93/22680, PCT/US93/04145, PCT/US93/08015, PCT/US94/07106, PCT/US94/12305, PCT/GB95/00542, PCT/US95/07377, PCT/US95/02024, PCT/US96/05480, PCT/US96/11147, PCT/US96/14839, PCT/US96/15606, PCT/US97/01603, PCT/US97/02102, PCT/GB97/005566, PCT/US97/06535, PCT/GB97/01148, PCT/GB97/01258, PCT/US97/08319, PCT/US97/08446, PCT/US97/10365, PCT/US97/17002, PCT/US97/16738, PCT/US97/19665, PCT/US97/20313, PCT/US97/21209, PCT/US97/21782, PCT/US97/23360, PCT/US98/06414, PCT/US98/01206, PCT/GB98/00975, PCT/US98/04280, PCT/US98/04571, PCT/US98/05438, PCT/US98/05451, PCT/US98/12442, PCT/US98/12779, PCT/US98/12930, PCT/US98/13949, PCT/US98/15151, PCT/US98/15469, PCT/US98/15458, PCT/US98/15456, PCT/US98/16971, PCT/US98/16686, PCT/US99/19069, PCT/US98/18873, PCT/US98/18541, PCT/US98/19325, PCT/US98/22966, PCT/US98/26925, PCT/US98/27405 and PCT/IB99/00048, all of which are incorporated by reference in their entireties for all purposes. All the above cited patent applications and other references cited throughout this specification are incorporated herein by reference in their entireties for all purposes.

The embodiments of the invention will be described using GeneChip® high oligonucleotide density probe arrays (available from Affymetrix, Inc., Santa Clara, Calif., USA) as exemplary embodiments. One of skill the art would appreciate that the embodiments of the invention are not limited to high density oligonucleotide probe arrays. In contrast, the embodiments of the invention are useful for analyzing any parallel large scale biological analysis, such as those using nucleic acid probe array, protein arrays, etc.

Gene expression monitoring using GeneChip® high density oligonucleotide probe arrays are described in, for example, Lockhart et al., 1996, Expression Monitoring By Hybridization to High Density Oligonucleotide Arrays, Nature Biotechnology 14:1675-1680; U.S. Pat. Nos. 6,040,138 and 5,800,992, all incorporated herein by reference in their entireties for all purposes.

In the preferred embodiment, oligonucleotide probes are synthesized directly on the surface of the array using photolithography and combinatorial chemistry as disclosed in several patents previous incorporated by reference. In such embodiments, a single square-shaped feature on an array contains one type of probe. Probes are selected to be specific against desired target. Methods for selecting probe sequences are disclosed in, for example, U.S. patent application Ser. Nos. ______, Attorney Docket Number 3359; ______, filed Nov. 21, 2000, Attorney Docket Number 3367, filed Nov. 21, 2000, and ______, Attorney Docket Number 3373, filed Nov. 21, 2000, all incorporated herein by reference in their entireties for all purposes.

In a preferred embodiment, oligonucleotide probes in the high density array are selected to bind specifically to the nucleic acid target to which they are directed with minimal non-specific binding or cross-hybridization under the particular hybridization conditions utilized. Because the high density arrays of this invention can contain in excess of 1,000,000 different probes, it is possible to provide every probe of a characteristic length that binds to a particular nucleic acid sequence. Thus, for example, the high density array can contain every possible 20 mer sequence complementary to an IL-2 mRNA. There, however, may exist 20 mer subsequences that are not unique to the IL-2 mRNA. Probes directed to these subsequences are expected to cross hybridize with occurrences of their complementary sequence in other regions of the sample genome. Similarly, other probes simply may not hybridize effectively under the hybridization conditions (e.g., due to secondary structure, or interactions with the substrate or other probes). Thus, in a preferred embodiment, the probes that show such poor specificity or hybridization efficiency are identified and may not be included either in the high density array itself (e.g., during fabrication of the array) or in the post-hybridization data analysis.

Probes as short as 15, 20, 25 or 30 nucleotides are sufficient to hybridize to a subsequence of a gene and that, for most genes, there is a set of probes that performs well across a wide range of target nucleic acid concentrations. In a preferred embodiment, it is desirable to choose a preferred or “optimum” subset of probes for each gene before synthesizing the high density array.

In some preferred embodiments, the expression of a particular transcript may be detected by a plurality of probes, typically, up to 5, 10, 15, 20, 30 or 40 probes. Each of the probes may target different sub-regions of the transcript. However, probes may overlap over targeted regions.

In some preferred embodiments, each target sub-region is detected using two probes: a perfect match (PM) probe that is designed to be completely complementary to a reference or target sequence. In some other embodiments, a PM probe may be substantially complementary to the reference sequence. A mismatch (MM) probe is a probe that is designed to be complementary to a reference sequence except for some mismatches that may significantly affect the hybridization between the probe and its target sequence. In preferred embodiments, MM probes are designed to be complementary to a reference sequence except for a homomeric base mismatch at the central (e.g., 13^{th} in a 25 base probe) position. Mismatch probes are normally used as controls for cross-hybridization. A probe pair is usually composed of a PM and its corresponding MM probe. The difference between PM and MM provides an intensity difference in a probe pair.

II. Data Analysis Systems

In one aspect of the invention, methods, computer software products and systems are provided for computational analysis of microarray intensity data for determining the presence or absence of genes in a given biological sample. Accordingly, the present invention may take the form of data analysis systems, methods, analysis software, etc. Software written according to the present invention is to be stored in some form of computer readable medium, such as memory, or CD-ROM, or transmitted over a network, and executed by a processor. For a description of basic computer systems and computer networks, see, e.g., Introduction to Computing Systems: From Bits and Gates to C and Beyond by Yale N. Patt, Sanjay J. Patel, 1st edition (Jan. 15, 2000) McGraw Hill Text; ISBN: 0072376902; and Introduction to Client/Server Systems: A Practical Guide for Systems Professionals by Paul E. Renaud, 2nd edition (Jun. 1996), John Wiley & Sons; ISBN: 0471133337.

Computer software products may be written in any of various suitable programming languages, such as C, C++, C# (Microsoft®), Fortran, Perl, MatLab (MathWorks, www.mathworks.com), SAS, SPSS and Java. The computer software product may be an independent application with data input and data display modules. Alternatively, the computer software products may be classes that may be instantiated as distributed objects. The computer software products may also be component software such as Java Beans (Sun Microsystem), Enterprise Java Beans (EJB, Sun Microsystems), Microsoft® COM/DCOM (Microsoft®), etc.

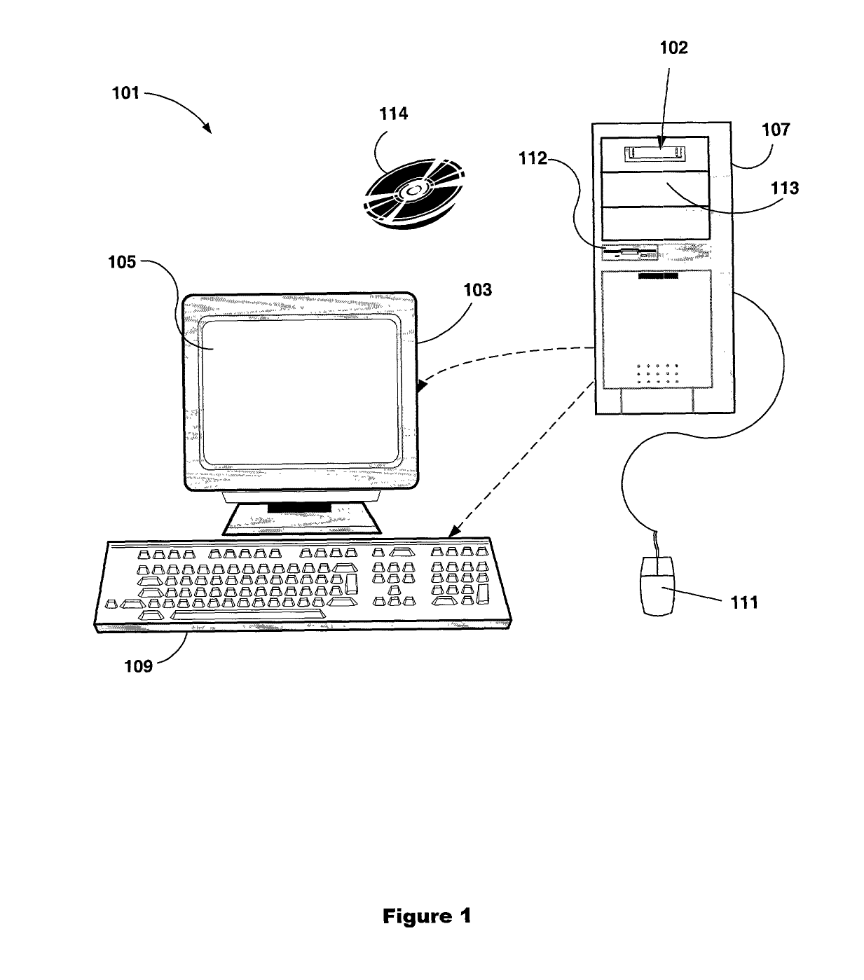

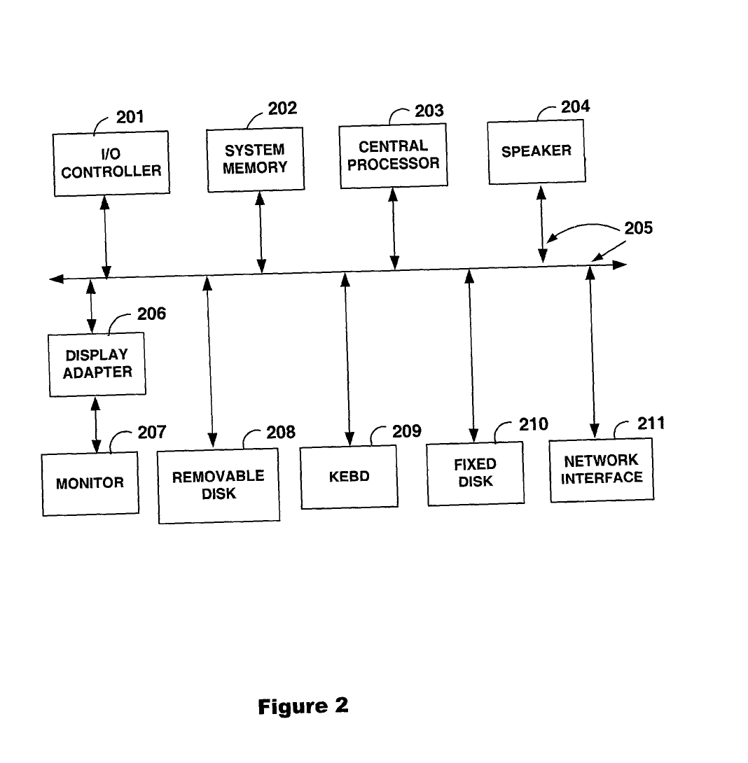

FIG. 1 illustrates an example of a computer system that may be used to execute the software of an embodiment of the invention. The computer system described herein is also suitable for hosting a DBMS. FIG. 1 shows a computer system 101 that includes a display 103, screen 105, cabinet 107, keyboard 109, and mouse 111. Mouse 111 may have one or more buttons for interacting with a graphic user interface. Cabinet 107 houses a floppy drive 112, CD-ROM or DVD-ROM drive 102, system memory and a hard drive (113) (see also FIG. 2) which may be utilized to store and retrieve software programs incorporating computer code that implements the invention, data for use with the invention and the like. Although a CD 114 is shown as an exemplary computer readable medium, other computer readable storage media including floppy disk, tape, flash memory, system memory, and hard drive may be utilized. Additionally, a data signal embodied in a carrier wave (e.g., in a network including the Internet) may be the computer readable storage medium.

FIG. 2 shows a system block diagram of computer system 101 used to execute the software of an embodiment of the invention. As in FIG. 1, computer system 101 includes monitor 201, and keyboard 209. Computer system 101 further includes subsystems such as a central processor 203 (such as a Pentium™ III processor from Intel), system memory 202, fixed storage 210 (e.g., hard drive), removable storage 208 (e.g., floppy or CD-ROM), display adapter 206, speakers 204, and network interface 211. Other computer systems suitable for use with the invention may include additional or fewer subsystems. For example, another computer system may include more than one processor 203 or a cache memory. Computer systems suitable for use with the invention may also be embedded in a measurement instrument.

III. Expression Constraint Analysis by Linear Programming Estimator

In one aspect of the invention, methods, systems and computer software products are provided for gene expression data analysis. The methods are based on constraining possible expression levels using simple models.

The embodiments of the invention are particularly useful for analyzing results of nucleic acid probe array based gene expression experiments where probes generally hybridize linearly with their targets; where the major error is cross hybridization; where hybridization intensities are positive and continuous quantities; where relative few probe suffer death, saturation, or irregular noise and where chip effects are multiplicative changes to the scale of the intensities. In such embodiments, the intensity (I) of a probe may be decomposed to:

I=S·C·T+H (1)

where: S is chip scale (to adjust for variations among chips);

C is coupling between the level of the targeted transcript in the sample and the intensity; T is the relative level of the transcript; and H is the effect of cross hybridization.

While effect of cross hybridization on intensity is generally unknown, it is greater than zero. Therefore:

I≧S·C·T (2)

or

log(I)≧log(S)+log(C)+log(T) (3)

Linear programming may be used to maximize the true effect and obtain estimates of the parameters including T, the relative level of the transcript in a sample.

Some embodiments of the methods of the invention will be described using the following notations. One of skill in the art would appreciate that the methods of the invention are not limited to the specific notations used herein. Rather, the notations are used for the purpose of describing embodiments of the invention.

s(i) is log(S) for the ith chip; c(j, k) is the coupling (log(C)) for jth probe and kth transcript; and x(k, l) is the log(T) for the kth transcript in the lth sample. Y(i, j, k, l) is the log(I) for jth probe for kth transcript in the ith chip hybridized with the lth sample. With the notations, Equation 3 may be written as follows:

Y(i,j,k,l)≧s(i)+c(j,k)+x(k,l) (4)

The parameters may be estimated by maximizing Σ(s(i)+c(j,k)+x(k,l)) (i.e., maximizing the true effect). Alternatively, the parameters may also be estimated by minimizing Σ(Y(i,j,k,l)−s(i)−c(j,k)−x(k,l)). Because x(k,l) is the log(relative transcript level), Σx(k,l)=0. Since Σx(k,l)=0, this may be equivalent to maximize Σ(s(i)+c(j,k)). In some embodiments, the chip effect, s(i) may be estimated independently, for example, by spiking each chip with known concentration of a control transcript or by using normalization controls such as probes against maintenance genes. Exemplary methods for estimating normalization factor to account for chip to chip variation are disclosed in, for example, U.S. patent application Ser. No. ______, Attorney Docket Number 3364, filed on Dec. 12, 2000, which is incorporated herein in its entirety by reference for all purposes.

In such embodiments, Σc(j,k) is maximized, i.e., maximizing the probe effects due to the true target. In some embodiments, where target transcripts are measured using perfect match (PM) and mismatch probes (MM), the additional constraints that c(PM)>c(MM) may be added. Additional constraints, such as those derived mixed samples, replicates, dilutions, or other modifications, may also be added.

Computer software code examples suitable for performing linear programming analysis are provided in, for example, the Numerical Recipes (NR) books developed by Numerical Recipes Software and published by Cambridge University Press (CUP, with U.K. and U.S. web sites).

One important estimator of the linear programming operation with the constraints described above is the x(k,l) or exp(x(k,l)), i.e., the relative quantities of transcript k in the sample relative to others in the experiments in the data set.

In an exemplary data set with 49 chips, 400,000 active probes, measuring 490,000 transcripts, there will be 49+400,000−490,000=790,000 variables and 19,600,000 constraints.

If s(i) is independently estimated, the problem is much easier to solve. In a data set of 100 chips with one million probes each, the program has 100 million constraints on ten million variables (if 10 probes/gene). However, if s(i) is estimated independently, the problem is simplified into estimating independent transcript/probe effects, which are much easier to solve, i.e., 100,000 instances of 1000 constraints on 110 variables.

Since the methods of the invention explicitly fit a model, residuals are acquired during the process, which may be permuted and re-sampled in a number of ways to produce confidence intervals by standard techniques, particularly computer intensive statistical inference procedures. Computer intensive statistical inference procedures are described in, e.g., Edgington, E. S. (1987). Randomization tests (2^{nd} Ed.). New York; Marcel Dekker. Efron, B. (1982)The Jackknife, the Bootstrap and other resampling plans. Philadelphia: Society for Industrial and Applied mathematics; Efron, B., & Tibshirani, R. J. (1993). An introduction to the bootstrap. New York: Chapman & Hall; Good, P. (1994). Permutation tests: A practical guide to resampling methods for testing hypotheses. New York: Springer-Verlag New York; Ludbrook, J., & Dudley, H. (1998). Why permutation tests are superior to t and F tests in biomedical research. The American Statistician, 52(2), 127-132; Manly, B. F. J. (1991). Randomization and Monte Carlo methods in biology. London, U.K.: Chapman & Hall; Mooney, C. Z., & Duval, R. D. (1993). Bootstrapping: A nonparametric approach to statistical inference. Newbury Park, Calif.: Sage Publications; Noreen, E. W. (1989). Computer intensive methods for testing hypotheses: An introduction. New York: John Wiley & Sons; Seltzer, M. H. (1993). Sensitivity analysis for fixed effects in the hierarchical model: A Gibbs sampling approach. Journal of Educational Statistics, 18(3), 207-235, all incorporated herein by reference in their entireties for all purposes.

Bootstrapping procedures use resampling with replacement from an already-drawn sample. Efron & Tibshirani (1993, previously incorporated by reference) provide the generic algorithm for performing a bootstrapping procedure as follows:

1. Draw a random “bootstrap” sample of size n with replacement (i.e., an observation, once drawn, may be drawn again), and calculate the “bootstrap” statistic of interest from this sample.

2. Repeat step (1) a large number N of times.

3. Estimate the “bootstrap standard error” of the parameter of interest using the N bootstrap statistics as the inputs for the usual standard error equation.

A shortcoming of bootstrapping is that all methods for estimating bootstrap confidence intervals rely to some degree on either the normal or t-distribution (Efron & Tibshirani, 1993). For N reasonably large, however, this should not pose a problem, even for relatively small sample sizes (Mooney & Duval, 1993). The “Jackknife” procedure is a special case of the bootstrap. Mooney & Duval (1993, previously incorporated by reference) provided algorithm for performing a jackknife procedure.

There are a number of different ways to obtain confidence interval by re-sampling. For example, residuals across experiments within the data points for a single probe may be re-sampled (under the worst-case assumption that probes still behave differently after factoring out first-order effects). Residuals across experiments within the data points associated with a single transcript may be re-sampled (under the assumption that transcript-level interactions are unique). Residuals across chips (under the assumption that chip-sample interactions are unique) may be resampled. Residuals across everything (since the first order effects of chip, transcript, and probe are factored out, everything else is assumed exchangeable) may be resampled. Resample from the lowest intensity values in the near vicinity of each probe, without re-fitting parameters may be performed to estimate the ‘background’ level of estimation of a transcript (assuming that low-intensity probes are drawn from a sufficiently similar distribution in the near vicinity to give an estimate of background). In such embodiments, re-sampling yields a confidence interval for background (i.e., zero transcript present), as opposed to the point estimate given by background subtraction). In some embodiments, a transcript is called as ‘absent’ when the confidence interval for background contains the estimator for the transcript level.

### CONCLUSION

The present inventions provide methods and computer software products for analyzing gene expression profiles. It is to be understood that the above description is intended to be illustrative and not restrictive. Many variations of the invention will be apparent to those of skill in the art upon reviewing the above description. By way of example, the invention has been described primarily with reference to the use of a high density oligonucleotide array, but it will be readily recognized by those of skill in the art that other nucleic acid arrays, other methods of measuring transcript levels and gene expression monitoring at the protein level could be used. The scope of the invention should, therefore, be determined not with reference to the above description, but should instead be determined with reference to the appended claims, along with the full scope of equivalents to which such claims are entitled.

All cited references, including patent and non-patent literature, are incorporated herewith by reference in their entireties for all purposes.

What is claimed is:

1. A method for analyzing plurality of transcripts in a plurality of samples using a plurality of nucleic acid probe arrays comprising:
a) obtaining a plurality of intensities, each of which reflects the hybridization of one of a plurality of probes in the plurality of samples; and
b) determining the couplings between the level of the transcript and the intensities, relative transcript levels and scales of probe arrays by minimizing the effect of cross-hybridization using linear programming with the constraint that the effect of cross-hybridization is non-zero.

2. The method of claim 1 wherein the minimizing comprising maximizing Σ(s(i)+c(j,k)+x(k,l)) or minimizing Σ(Y(i,j,k,l)−s(i)−c(j,k)−x(k,l)) with the constraint Y(i,j,k,l)≧s(i)+c(j,k)+x(k,l), wherein s(i) is log(scale of probe array) for the ith probe array, c(j, k) is the (log(the coupling between transcript and intensity)) for jth probe and kth transcript, x(k, l) is the log(relative transcript level) for the kth transcript in the lth sample, and Y(i, j, k, l) is the log(I) for jth probe for kth transcript in the ith probe array hybridized with the lth sample.

3. The method of claim 2 wherein Σ(s(i)+c(j,k)+x(k,l)) is equivalent to Σ(s(i)+c(j,k)) and Σx(k,l)=0.

4. The method of claim 3 wherein the maximizing or minimizing is further constrained by coupling for perfect match probes is greater than that for mismatch probes.

5. The method of claim 4, wherein the scale of probe array is determined independent of the maximizing.

6. The method of claim 5 wherein the probe array effect is determined using normalization probes on the probe arrays.

7. The method of claim 1 further comprising determining confidence intervals for the relative transcript levels, couplings and scales by bootstrapping on residues, probe arrays or probes.

8. A system for analyzing plurality of transcripts in a plurality of samples using a plurality of nucleic acid probe arrays comprising:
a processor; and
a memory being coupled with the processor; the memory storing a plurality of machine instructions that cause the processor to perform a plurality of steps when implemented by the processor, the logical steps comprising:
obtaining a plurality of intensities, each of which reflects the hybridization of one of a plurality of probes in the plurality of samples; and
determining the couplings between the level of the transcript and the intensities, relative transcript levels and scales of probe arrays by minimizing the effect of cross-hybridization using linear programming with the constraint that the effect of cross-hybridization is non-zero.

9. The system of claim 8 wherein the minimizing comprising maximizing Σ(s(i)+c(j,k)+x(k,l)) or minimizing Σ(Y(i,j,k,l)−s(i)−c(j,k)−x(k,l)) with the constraint Y(i,j,k,l)≧s(i)+c(j,k)+x(k,l), wherein s(i) is log(scale of probe array) for the ith probe array, c(j, k) is the (log(the coupling between transcript and intensity)) for jth probe and kth transcript, x(k, l) is the log(relative transcript level) for the kth transcript in the lth sample, and Y(i, j, k, l) is the log(I) for jth probe for kth transcript in the ith probe array hybridized with the lth sample.

10. The system of claim 9 wherein Σ(s(i)+c(j,k)+x(k,l)) is equivalent to Σ(s(i)+c(j,k)) and Σx(k,l)=0.

11. The system of claim 10 wherein the maximizing or minimizing is further constrained by coupling for perfect match probes is greater than that for mismatch probes.

12. The system of claim 11 wherein the scale of probe array is determined independent of the maximizing.

13. The system of claim 12 wherein the probe array effect is determined using normalization probes on the probe arrays.

14. The system of claim 11 further comprising determining confidence intervals for the relative transcript levels, couplings and scales by bootstrapping on residues, probe arrays or probes.

15. A computer readable medium having computer executable instructions for performing a method comprising:
obtaining a plurality of intensities, each of which reflects the hybridization of one of a plurality of probes in the plurality of samples; and
determining the couplings between the level of the transcript and the intensities, relative transcript levels and scales of probe arrays by minimizing the effect of cross-hybridization using linear programming with the constraint that the effect of cross-hybridization is non-zero.

16. The computer readable medium of claim 15 wherein the minimizing comprising maximizing Σ(s(i)+c(j,k)+x(k,l)) or minimizing Σ(Y(i,j,k,l)−s(i)−c(j,k)−x(k,l)) with the constraint Y(i,j,k,l)≧s(i)+c(j,k)+x(k,l), wherein s(i) is log(scale of probe array) for the ith probe array, c(j, k) is the (log(the coupling between transcript and intensity)) for jth probe and kth transcript, x(k, l) is the log(relative transcript level) for the kth transcript in the lth sample, and Y(i, j, k, l) is the log(I) for jth probe for kth transcript in the ith probe array hybridized with the lth sample.

17. The computer readable medium of claim 16 wherein Σ(s(i)+c(j,k)+x(k,l)) is equivalent to Σ(s(i)+c(j,k)) and Σx(k,l)=0.

18. The computer readable medium of claim 17 wherein the maximizing or minimizing is further constrained by coupling for perfect match probes is greater than that for mismatch probes.

19. The computer readable medium of claim 17 wherein the scale of probe array is determined independent of the maximizing.

20. The computer readable medium of claim 19 wherein the probe array effect is determined using normalization probes on the probe arrays.

21. The computer readable medium of claim 20 further comprising determining confidence intervals for the relative transcript levels, couplings and scales by bootstrapping on residues, probe arrays or probes.
